(19) United States
(12) Patent Application Publication
(10) Pub. No.: US 2010/0003384 A1
(43) Pub. Date: Jan. 7, 2010

(54) EFFERVESCENT POWDER FORMULATION FOR BEVERAGES AND ITS USE

(76) Inventors: Aires Iacovone, Sao Paulo (BR); Maisa Peixoto Munhoz, Paulinia (BR); Alessandro Rizzato, Sao Paulo (BR)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 12/305,050

(22) PCT Filed: Jun. 22, 2007
(86) PCT No.: PCT/BR2007/000164

§ 371 Date: May 19, 2009

(30) Foreign Application Priority Data
Jun. 23, 2006 (BR) PI0602825-0

(51) Int. Cl. A23L 2/40; A23L 2/68; A23L 2/52; A61K 9/46

(52) U.S. Cl. 426/285; 426/591; 426/477; 426/443

(57) ABSTRACT

Effervescent powder formulation having self-refrigerant power that consists of a mixture of sodium bicarbonate, citric acid, and tartaric acid, useful for obtaining an instant sparkling beverage

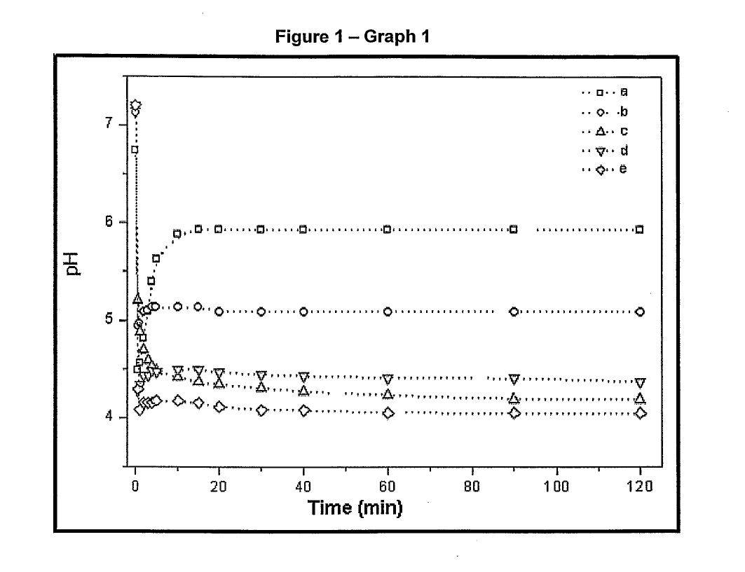

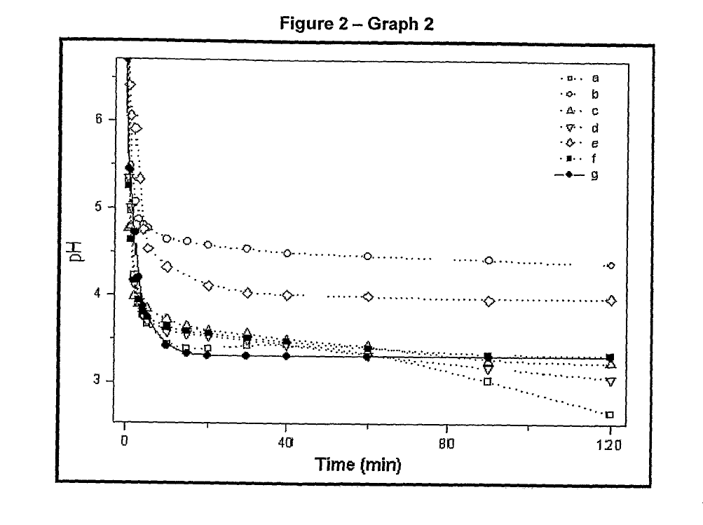

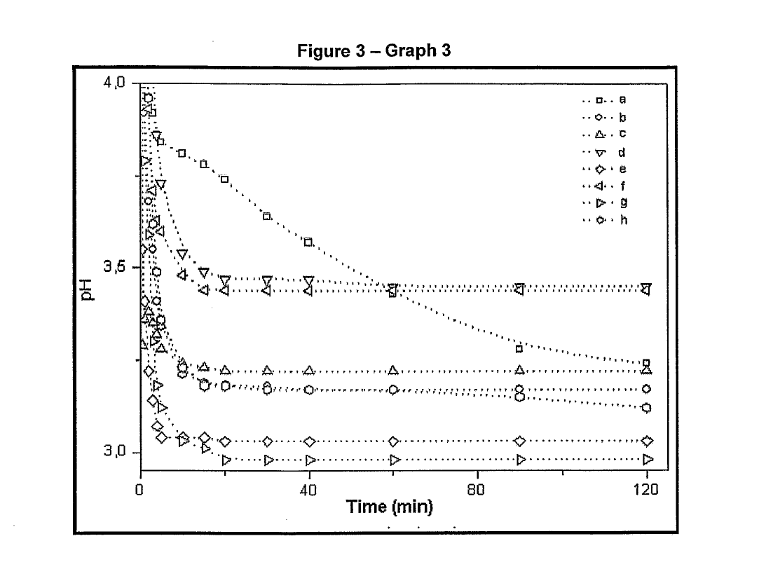

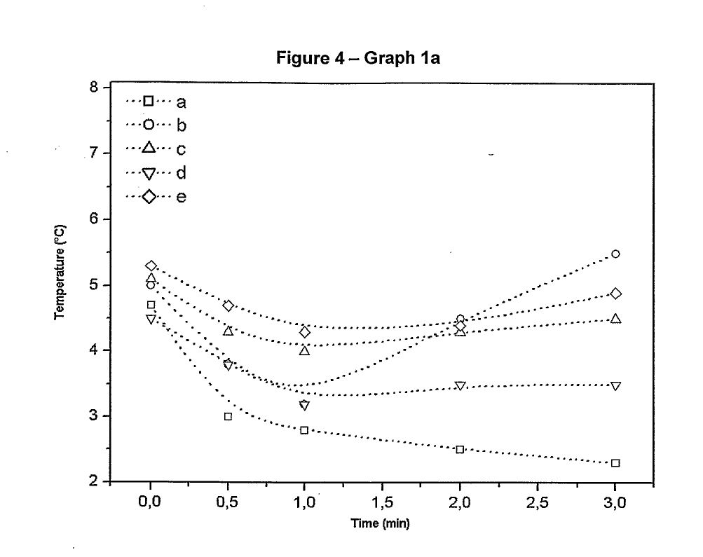

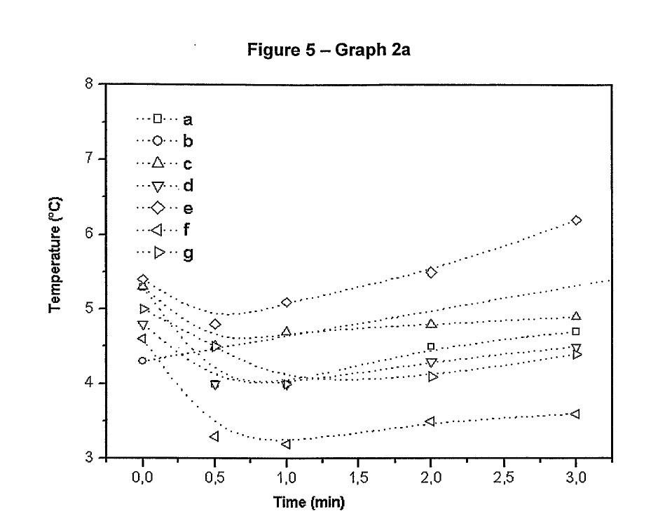

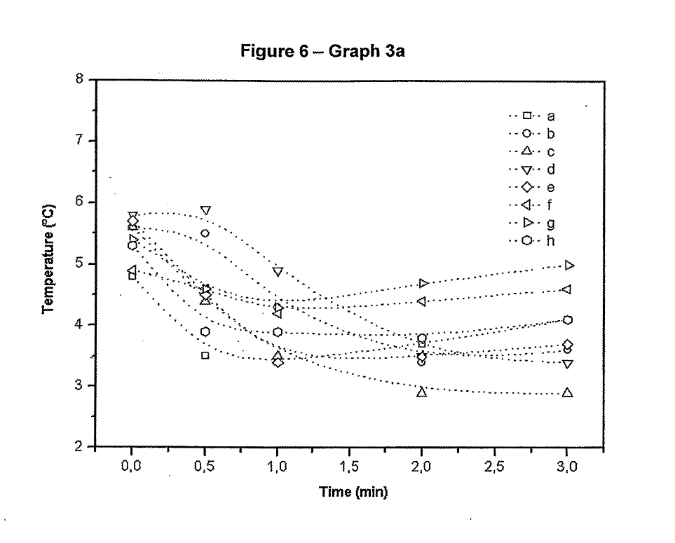

### FIELD OF THE INVENTION

The present invention refers to a self-refrigerant effervescent powder formulation for beverages, to obtain an instant sparkling beverage when mixed with water or juice.

### BACKGROUND OF THE INVENTION

The art of making effervescent beverages has been in existence since the 19^{th} Century. North-American patent U.S. Pat. No. 541,255, dating from 1895, had already disclosed the invention of an effervescent beverage, based on a mixture of granules containing alkaline bicarbonate and, separately, an organic acid coated with sugar. According to this document, when put in water, the granules have the appearance and taste of champagne, and the effervescent effect only lasts from ten to thirty minutes.

One of the problems in making effervescent beverages is the release of carbon gas, which is invariably short-lasting, as in the above-cited reference. This is why a greater amount of bicarbonate is added, and consequently a greater amount of acid, in order to obtain a prolonged effect of release of carbon gas. However, with an increase in the amount of bicarbonate, the pH value of the solution tends to increase over time, giving the solution a saline taste.

Another example relating to the manufacture of effervescent beverages is the mixture of an alkaline bicarbonate with just one organic acid, for example, sodium bicarbonate and citric acid, or sodium bicarbonate and malic acid, as can be seen in patent document JP60166376, which describes an effervescent beverage made from a combination of a bicarbonate with an acid, with a self-refrigerant characteristic.

In North-American patent U.S. Pat. No. 3,660,107 the problem referred to of the saline taste of bicarbonate is addressed by using a combination of two organic acids with alkaline bicarbonate, these being tartaric acid, citric acid, and sodium bicarbonate, at a ratio of 2:1:4, respectively. However, as can be seen, the amount of sodium bicarbonate is even greater than that of the acids, because it was believed that the amount of sodium bicarbonate was responsible for the release of gas into the solution.

An innovative technique was developed in this invention to be used in the manufacture of effervescent powder products for beverages, which keeps the acid pH of the solution substantially constant, with prolonged release of gas, in addition to the self-refrigerant power.

### DESCRIPTION OF THE INVENTION

All percentage amounts stated herein refer to the percentage in relation to the total weight of the formulation of the invention.

The invention consists of the combination of an alkaline bicarbonate or alkaline earth with two different organic acids, acceptable for human consumption, the amount of bicarbonate being lower or equal to the largest amount of one or the other acid. This combination uses a lower amount of bicarbonate than that found in the state of the art, and limited amounts of acids, ensuring that no increase in pH value occurs, and, consequently, avoiding the need for a greater amount of acids to obtain a prolonged release of carbon gas and a temperature decrease of the solution.

The contents of the above combination, as per the invention formulation, vary between about 1.89% and about 8.5%.

Suitable bicarbonates for the formulation of the present invention are sodium bicarbonate, calcium bicarbonate, potassium bicarbonate, barium bicarbonate, strontium bicarbonate, and magnesium bicarbonate. In particular, without excluding any others, sodium bicarbonate is used.

The expression referring to acids that are “acceptable for human consumption” indicates any acid material having pKₐ of at least about 5, preferably in the range between 2 and 5, considered safe for ingestion by humans. Some more common examples of acids, without excluding any others, are citric, ascorbic, malic, succinic, tartaric, phosphoric, and monopotassic phosphate. Particularly suitable are organic acids such as citric, tartaric and malic, preferably citric and tartaric.

In a specific embodiment, the invention consists of a combination of sodium bicarbonate with citric and tartaric acids. Suitably, the amount of bicarbonate is less than or equal to 3%, that of citric acid is less than or equal to 2.5%, and that of tartaric acid is less than or equal to 3%. In particular, sodium bicarbonate may vary between 0.88% and 2.5%, citric acid between 0.05% and 2.5% and tartaric acid between 0.88% and 3%.

Suitable ratios of sodium bicarbonate, citric acid, and tartaric acid in this present invention are, respectively:

2:1:3

2.5:2:2.5

2.5:2.5:2

2.5:2:3

1:0.05:1

1:1:1

The pH value obtained by the invention formulation when dissolved in water is substantially constant for at least 1 hour, particularly for at least 2 hours. Said pH value is less than 4, particularly less than 3.5 and more particularly less than 3.

The release of carbon gas obtained by dissolving the invention formulation in water occurs over at least 1 hour, particularly over at least two 2 hours.

The invention formulation is able to lower the temperature from between 1° C. and 10° C. of the beverage obtained by dissolving it in water, the temperature of which is, at most, environment temperature, particularly less than 10° C. or less than 5° C. The temperature drop occurs in up to around two minutes after contact of the formulation with the liquid.

Particularly, a suitable minimum percentage ratio of the combination of sodium bicarbonate, citric acid and tartaric acid is 0.88%:0.05%:0.88%, in which the combination has a pH value of around 3.5, considered ideal for soft-drink beverages, without having the saline taste that is common in basic pH and it is also not too acidic to the point of making the beverage taste unpleasant.

The invention formulation is in solid form, for example, in powder, granule, or tablet form. It is used to obtain a beverage that self-cools and releases gas when dissolved in liquids suitable for ingestion, for example, water, natural fruit juices, etc.

The invention formulation may contain other additives, for example, one or more sweeteners, sugars, anti-humectants, colorants, flavorings, aromatizers, etc.

The graphs below show the advantages of the combination of three compounds.

Graphs 1 and 2 disclose solutions known in the art, whereas graph 3 refers to the present invention.

### DESCRIPTION OF THE DRAWINGS

Graph 1 illustrates the variation of pH value in relation to time, for a combination of sodium bicarbonate and citric acid only. Each letter corresponds to a different ratio:

a. 4% sodium bicarbonate and 3% citric acid;
b. 3% sodium bicarbonate and 3% citric acid;
c. 2% sodium bicarbonate and 3% citric acid;
d. 3% sodium bicarbonate and 4% citric acid;
e. 2.5% sodium bicarbonate and 4% citric acid.

Under these conditions, the pH value varies between 6 and 4, and rarely remains constant.

Graph 2 illustrates the variation of the pH value in relation to time, for a combination of sodium bicarbonate and tartaric acid only. Each letter corresponds to a different ratio:

a. 2% sodium bicarbonate and 3.5% tartaric acid;
b. 3% sodium bicarbonate and 3% tartaric acid;
c. 2% sodium bicarbonate and 3% tartaric acid;
d. 1.75% sodium bicarbonate and 3% tartaric acid;
e. 2.25% sodium bicarbonate and 2.5% tartaric acid;
f. 2.5% sodium bicarbonate and 4% tartaric acid;
g. 2.5% sodium bicarbonate and 3.5% tartaric acid.

Again, it is possible to see the difficulty in keeping the pH value constant. In this case, the mixture is more acidic as a consequence of the decrease in the amount of sodium bicarbonate.

Graph 3 illustrates the variation of the pH value in relation to time, for a combination of sodium bicarbonate, citric acid, and tartaric acid. Each letter corresponds to a different ratio:

a. 2.5% sodium bicarbonate, 2% citric acid and 2% tartaric acid;
b. 2.5% sodium bicarbonate, 2% citric acid and 2.5% tartaric acid;
c. 2.5% sodium bicarbonate, 2.5% citric acid and 2% tartaric acid;
d. 3% sodium bicarbonate, 2% citric acid and 2.5% tartaric acid;
e. 2.5% sodium bicarbonate, 2% citric acid and 3% tartaric acid;
f. 2.5% sodium bicarbonate, 1% citric acid and 2% tartaric acid;
g. 2% sodium bicarbonate, 1% citric acid and 3% tartaric acid;
h. 2.25% sodium bicarbonate, 1% citric acid and 3% tartaric acid.

It can be seen that in the combinations where the amount of bicarbonate is less than or equal to the greater amount of one of the acids, there is an increase in the acidity together with greater stability. Said formulation, when dissolved in water, also produces a more prolonged release of gas for at least 2 hours and a pH value less than 4 substantially constant for at least two hours.

Graph 1a illustrates the variation in temperature in relation to time, for a combination of sodium bicarbonate and citric acid only. Each letter corresponds to a different ratio:

a. 4% sodium bicarbonate and 3% citric acid;
b. 3% sodium bicarbonate and 3% citric acid;
c. 2% sodium bicarbonate and 3% citric acid;
d. 3% sodium bicarbonate and 4% citric acid;
e. 2.5% sodium bicarbonate and 4% citric acid.

In these conditions, the temperature shows a drop before two minutes, but after two minutes it becomes inconstant and rises.

Graph 2a illustrates the variation in temperature in relation to time, for a combination of sodium bicarbonate and tartaric acid only. Each letter corresponds to a different ratio:

a. 2% sodium bicarbonate and 3.5% tartaric acid;
b. 3% sodium bicarbonate and 3% tartaric acid;
c. 2% sodium bicarbonate and 3% tartaric acid;
d. 1.75% sodium bicarbonate and 3% tartaric acid;
e. 2.25% sodium bicarbonate and 2.5% tartaric acid;
f. 2.5% sodium bicarbonate and 4% tartaric acid;
g. 2.5% sodium bicarbonate and 3.5% tartaric acid.

Once again, it can be noted that despite the drop in temperature before two minutes, it becomes inconstant and soon rises.

Graph 3a illustrates the variation in temperature in relation to time, for a combination of sodium bicarbonate, citric acid, and tartaric acid. Each letter corresponds to a different ratio:

a. 2.5% sodium bicarbonate, 2% citric acid and 2% tartaric acid;
b. 2.5% sodium bicarbonate, 2% citric acid and 2.5% tartaric acid;
c. 2.5% sodium bicarbonate, 2.5% citric acid and 2% tartaric acid;
d. 3% sodium bicarbonate, 2% citric acid and 2.5% tartaric acid;
e. 2.5% sodium bicarbonate, 2% citric acid and 3% tartaric acid;
f. 2.5% sodium bicarbonate, 1% citric acid and 2% tartaric acid;
g. 2% sodium bicarbonate, 1% citric acid and 3% tartaric acid;
h. 2.25% sodium bicarbonate, 1% citric acid and 3% tartaric acid.

It can be seen that in the combination of sodium bicarbonate with both citric and tartaric acids, there was a marked drop in temperature in less than two minutes and the temperature remained constant after two minutes with greater ease.

### EXAMPLES

Below are illustrative examples of particular embodiments of the invention. They are not intended to establish any limits. Limits are only as defined in the claims attached hereto.

### Example 1

### Dissolving a Powder Form Combination of Sodium Bicarbonate, Citric Acid, and Tartaric Acid 2:1:3

A powder form combination was prepared of sodium bicarbonate, citric acid, and tartaric acid at a ratio of 2:1:3. This content was dissolved in cold water (5±2° C.)—the dissolution occurred in less than two minutes. A reading of the resulting solution's pH value was in the order of 3.0 after around 10 minutes after adding the formulation components to the water. The solution was kept still under environment conditions, that is, under normal temperature and pressure conditions, and the pH value remained constant for over 2 hours.

### Example 2

### Dissolving a Tablet Form Combination of Sodium Bicarbonate, Citric Acid, and Tartaric Acid 2:1:3

A table form combination was prepared of sodium bicarbonate, citric acid, and tartaric acid at a ratio of 2:1:3. This content was dissolved in cold water (5±2° C.)—the dissolution occurred in less than 5 minutes. A reading of the resulting solution's pH value was in the order of 3.5 after around 20 minutes after adding the formulation components to the water. The solution was kept still at environment conditions, that is, under normal temperature and pressure conditions, and the pH value remained constant for over 2 hours.

Based on the data and graphs set forth herein, a person skilled in the art will know how to carry out an amount of embodiments not expressly stated herein, but within the scope of the invention, the limits being established by the claims appended hereto, and with equivalent results.

1. An effervescent powder formulation comprising a combination of a sodium bicarbonate with citric acid and tartaric acid, suitable for human consumption, wherein the amount of sodium bicarbonate is less than or equal to the greatest amount of one of the acids and the combination of sodium bicarbonate, citric acid and tartaric acid is at a ratio of 0.88-3:0.05-2.5:0.88-3, respectively.

2. The formulation according to claim 1, wherein the combination of sodium bicarbonate with citric acid and tartaric acid comprises between about 1.89% and about 8.5% in weight, in relation to the total weight of said formulation.

3. The formulation according to claim 1, wherein the sodium bicarbonate is less than or equal to 3%, the citric acid is less than or equal to 2.5% and the tartaric acid is less than or equal to 3%.

4. The formulation according to claim 1, wherein the combination of sodium bicarbonate, citric acid and tartaric acid is in ratios chosen from among 2:1:3; 2.5:2:2.5; 2.5:2.5:2; 2.5:2:3; 1:0.05:1; and 1:1:1.

5. The formulation according to claim 1, wherein the combination of sodium bicarbonate, citric acid and tartaric acid are at a minimum percentage ratio of 0.88%:0.05%:0.88%, respectively.

6. The use of an effervescent powder formulation according to claim 1, and comprising the manufacturing of a beverage that provides an acid pH substantially constant in the liquid in which said formulation is dissolved.

7. The use of the formulation according to claim 6, wherein said pH remains substantially constant for between at least about 1 hour and about 2 hours.

8. The use of the formulation according to claim 6, wherein said acid pH is under 4.0.

9. The use of the formulation according to claim 8, wherein said acid pH is under 3.5.

10. The use of the formulation according to claim 9, wherein said acid pH is under 3.0.

11. The use of an effervescent powder formulation according to claim 1, and comprising the manufacturing of a beverage that provides a prolonged release of gas.

12. The use of the formulation according to claim 11, wherein said prolonged release of gas is at least between about 1 hour and about 2 hours.

13. The use of an effervescent powder formulation, according to claim 1, and comprising the manufacturing of a beverage that reduces the temperature of a liquid from 1° C. to 10° C. when dissolved therein.

14. The use of a formulation according to claim 13, wherein the start of said drop in temperature occurs up to about two minutes after contact of the formulation with the liquid.

15. The use of a formulation according to claim 14, wherein said liquid is water or fruit juice.

16. The use of a formulation according to claim 13, wherein the temperature of said liquid is greater than zero degrees Celsius and less than the environment temperature.

17. The use of a formulation according to claim 16, wherein the temperature of said water is greater than zero degrees Celsius and lower than about 10° C.

18. The use of a formulation according to claim 17, wherein the temperature of said water is greater than zero degrees Celsius and lower than 5° C.

19. The use of an effervescent powder formulation according to claim 1, and comprising the manufacturing of a product in a powder, granule, or tablet form soluble in water to make it drinkable.

20. The use of a formulation according to claim 19, wherein said product is in powder form.

21. The use of a formulation according to claim 19, wherein said product comprises one or more sweeteners, sugars, anti-humectants, colorants, flavorings, or aromatizers.

22. The use of an effervescent powder formulation, according to claim 1, and further comprising the manufacturing of a useful product that is soluble in water or juices.
